(19) United States
(12) Patent Application Publication
(10) Pub. No.: US 2007/0148053 A1
(43) Pub. Date: Jun. 28, 2007

(54) Genetic smell source (GSS)

(76) Inventor: Qing Tian, Shrewsbury, MA (US)

Correspondence Address: TIAN, QING, SHREWSBURY GREEN DR., 8-E, SHREWSBURY, MA, 01545, US

(21) Appl. No.: 11/319,123

(22) Filed: Dec. 27, 2005

(51) Int. Cl. A61L 9/03

(52) U.S. Cl. 422/124; 422/125

(57) ABSTRACT

A genetic smell source (GSS) is provided. The genetic smell source (GSS) comprises a housing case and a USB drive circuit. The housing case farther comprises a perfume case and a perfume source. The USB drive circuit is housed inside the housing case. The USB drive circuit farther comprises a switch to provide operation interface to the user, a USB controller, and a micro controller for controlling the operation of the genetic smell source (GSS).

The present invention creates a concept for producing a mini aroma environment generated by a personal computer. The genetic smell source (GSS) can supply various smells with different smell source.

### FIELD OF THE INVENTION

The present invention generally relates to a Universal Serial Bus (USB) device, and more specifically to a genetic smell source (GSS) providing the functions of sending smell with digital device e.g. computer etc.

### BACKGROUND OF THE INVENTION

There are many home computer accessories in the market, but so far we have not seen any product connected the flavor with personal computer. There is a need for aroma generator controlled by computers: with the aroma generator, a pleasant environment for a user can be created. With the aroma generator, a special communication environment for online communicators can be created. With the aroma generator, a special treatment or providential environment can be created for health purpose.

The present invention creates a concept for producing a mini aroma environment generated by a personal computer. We have produced the pro-types for test purpose. The pro-type told us the idea can work properly with any type of computers. The pro-type also showed us that there're no any technical problems to produce them.

With the development of computer, people now can talk with each other and see each other, but none has been seen the effects of the smell. This third feeling sense can be added to the modern computer application environment.

### SUMMARY OF THE INVENTION

The present invention comprises a housing case and a USB drive circuit. The housing case farther comprises a perfume case and a perfume source. The USB drive circuit is housed inside the housing case. The USB drive circuit farther comprises a switch to provide operation interface to the user, a USB controller, and a micro controller for controlling the operation of the genetic smell source.

The foregoing and other objects, features, aspects and advantages of the present invention will become better understood from a careful reading of a detailed description provided herein below with appropriate reference to the accompanying drawings.

### BRIEF DESCRIPTION OF THE DRAWINGS

The present invention can be understood in more detail by reading the subsequent detailed description in conjunction with the examples and references made to the accompanying drawings, wherein:

FIG. 1 shows a sketch of a genetic smell source (GSS) of the present invention;

FIG. 2a shows a schematic view of a genetic smell source (GSS) of the present invention without USB cable;

FIG. 2b shows a schematic view of a genetic smell source (GSS) of the present invention with USB cable; and

FIG. 3 shows a block diagram of an embodiment of the USB drive circuit of the genetic smell source (GSS) of the present invention.

### DETAILED DESCRIPTION OF THE PREFERRED EMBODIMENTS

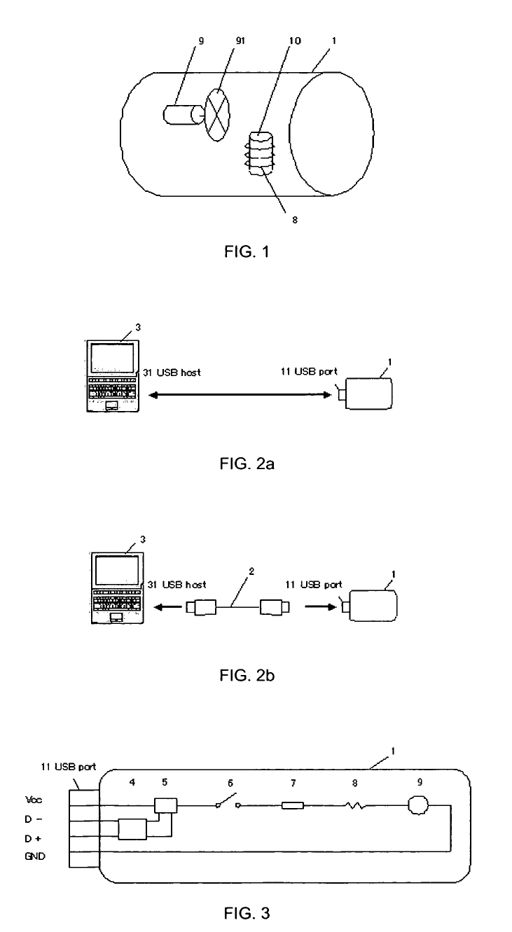

The genetic smell source (GSS) of the present invention is a USB device including a USB drive circuit housed in an external case. FIG. 1 shows the sketch of the genetic smell source (GSS) of the present invention. The block diagram of an embodiment of USB drive circuit is shown in FIG. 3.

FIG. 3 shows a block diagram of an embodiment of a USB drive circuit of the invention. USB drive circuit includes a USB bus power line, a housing case 1, a USB port 11, a USB controller 4, a micro controller 5, a switch 6, a light 7, a heater 8, a motor 9, USB controller 4, used for connecting to the USB host, such as computer, further includes a transceiver and serial interface engine (SIE) or parallel interface engine (PIE), and is responsible for the receiving and transmission of the USB specification version 1.1 or 2.0 protocol. USB controller 4 is coupled with micro controller 5. Switch 6 is for controlling the ON/OFF of light 7, heater 8 and motor 9. USB bus power line draws power from USB host and provides operation power to USB drive circuit. Micro controller 5 controls the operation of the entire drive circuit.

FIG. 1 shows a sketch of an embodiment of a genetic smell source (GSS). The sketch includes a housing case 1, a heater 8, a motor 9, a fan 91, and perfume source 10.

When the genetic smell source (GSS) is plugged into the USB host, user can use a switch and a software for the genetic smell source (GSS) to control the time of the ON/OFF of the genetic smell source (GSS).

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

Description of the Code

1. Housing case.
2. USB cable.
3. Computer.
4. USB controller.
5. Micro controller.
6. Switch.
7. Light.
8. Heater.
9. Motor.
10. Perfume body.
11. USB port.
31. USB host.
91. Fan.

What is claimed is:

1. A genetic smell source (GSS), comprising: a housing case; a USB drive circuit housed in said housing case; and a perfume source housed in said housing case.

2. The genetic smell source (GSS) as claimed in claim 1, wherein said housing case further comprises: a USB connector for connecting a USB host; and a perfume case for fixing a perfume source and the perfume case engaged to the said housing case.

3. The genetic smell source (GSS) as claimed in claim 1, wherein said USB drive circuit further comprises: a USB controller; a micro controller for controlling the operation of said genetic smell source (GSS); a switch for controlling the ON/OFF of said genetic smell source (GSS); a signal light; a heater for heating the perfume source; a motor for sending smell; a USB bus power line for drawing power from said USB host; and a USB cable.

4. The genetic smell source (GSS) as claimed in claim 3, wherein said USB controller further comprises a USB transceiver and a serial interface engine (SIE) for receiving and transmitting USB protocols.

5. The genetic smell source (GSS) as claimed in claim 3, wherein said USB controller further comprises a USB transceiver and a parallel interface engine (PIE) for receiving and transmitting USB protocols.

6. The genetic smell source (GSS) as claimed in claim 3, wherein said motor further comprises a fan.

7. The genetic smell source (GSS) comprising: a software of computer for controlling the genetic smell source (GSS).

8. The genetic smell source (GSS) comprising: using digital device e.g. computer etc.
